(19) United States
(12) Patent Application Publication
(10) Pub. No.: US 2011/0129858 A1
(43) Pub. Date: Jun. 2, 2011

(54) Prognosis Biomarker for Evaluating the Cure Level of Stroke Patient and a Method thereof

(75) Inventor: Chin-San Liu, Taichung City (TW)

(73) Assignee: CHANGHUA CHRISTIAN HOSPITAL, Changhua City (TW)

(21) Appl. No.: 12/626,805

(22) Filed: Nov. 27, 2009

(51) Int. Cl. G01N 33/53; C07K 14/475

(52) U.S. Cl. 435/7.92; 530/397

(57) ABSTRACT

A method for evaluating the cure level of a stroke patient comprises following steps: (1) obtaining isolated blood sample from said stroke patient; (2) determining the concentration of serum granulocyte colony-stimulating factor (G-CSF) of said blood sample; (3) comparing the relationship between said concentration of granulocyte colony-stimulating factor (G-CSF) and the stroke severity ranking of said stroke patient; wherein United State National Institute of Health Stroke Scale (NIHSS) or modified Ranking Scale (mRS) is used in said stroke severity ranking; and (4) Using said concentration of granulocyte colony-stimulating factor (G-CSF) to predict the possible cure level of said stroke patient. The invention further provides a prognosis biomarker for evaluating the cure level of a stroke patient, and a kit containing said prognosis biomarker.

### BACKGROUND OF THE INVENTION

1. Field of the Invention

The invention relates to a prognosis biomarker for evaluating the cure level of a stroke patient and a method thereof, and in particular, to endogenous granulocyte colony-stimulating factor (G-CSF) as a prognosis biomarker for evaluating the cure level of a stroke patient, and to a method for evaluating the cure level of a stroke patient by using the same.

2. Description of the Prior Art

The main cause of stroke is occlusion of blood vessel at various parts in the cerebrum, causing various degree of nerve function disorder such as abrupt deviation or paralysis of the body, blocked language, hands and feet not nimble or hand numbness, feet numbness and the like. At present, clinical therapy relies mostly on drug therapy such as administrating anti-coagulant, anti-platelet aggregating agent, and even thrombolytic agent. In these cases, a period of time after acute stage treatment might be needed to know whether the treatment is correct. In addition to common evaluating index such as reflex action, Glasgow coma scale (GCS), and the like, no other biomarker can be used to evaluate the prognosis in a short period. Mostly, the prognosis is poor, and places pressure on the relatives of the patient and can cost massive social resources as well. Prognosis is a medical term that a doctor uses to predict the disease progress of a patient, possible cure level (recovery extent), and to know whether the patient can be fully recovered or not.

Granulocyte colony-stimulating factor (G-CSF) is a hematopoietic growth factor, and is a glycoprotein synthesized in vascular endothelial cell, monocyte and fibroblast. The accession number of granulocyte colony-stimulating factor (G-CSF) in NCBI is REGION: 35425214 . . . 35427592 of NC_{—}000017 (http://www.ncbi.nlm.nih.gov/entrez/viewer.fcgi?val=NC_{—}000017.9&f rom=35425214&to =35427592&dopt=gb), and has a sequence as depicted in SEQ ID No: 1. Said gene (G-CSF) can be transcribed to three different mRNA variants that has accession numbers in NCBI as NM_{—}172220.1, NM_{—}000759.2, and NM_{—}172219.1, respectively.

Known functions of granulocyte colony-stimulating factor (G-CSF) can be described as followed:
1. Driving hematopoietic stem cell from rest stage to multiplication stage, and promoting the multiplication, maturation and release of neutrophilic granulocyte;
2. Enhancing functions of granulocyte (phagocytosis, chemotaxis), while giving little influence on other cells.

Recent clinical applications of granulocyte colony-stimulating factor (G-CSF) are mostly focused on disease study such as tumor chemotherapy, bone marrow graft, bone marrow suppression, AIDS, nosohemia, granulocyte deficiency, and the like; increasing leukocyte; and promoting infection resistance.

Subcutaneous injection of exogenous granulocyte colony stimulating-factor (G-CSF) to an occluded middle cerebral artery in recent rodent models of ischemic stroke both reduced infarction volume in the hyperacute stage (the neuroprotection effect) and enhanced functional recovery in subsequent subacute stages (the neurogenesis effect) (reference 1-5).

Human in vivo studies suggest that acute ischemic stroke and acute myocardial infarction (AMI) actually stimulate the endogenous release of G-CSF with serum levels peaking on day 2 and remain elevated for at least 6 days after the event (reference 6). In a small randomized control clinical trial, Shyu and colleagues demonstrated that the exogenous use of G-CSF within the first week following an ischemic stroke lead to a better prognosis and functional outcome than those in the placebo group (reference 7). Upon review of the literature, it became apparent that the relationship between endogenous G-CSF secretion and the prognosis and severity of ischemic stroke had not been reported before.

However, the inventor found that endogenous G-CSF might give positive influence on the stem cell during acute ischemic stroke. In addition, secretion of granulocyte colony-stimulating factor (G-CSF) is involved in the progress of human acute ischemic stroke. Accordingly, the aim of the invention is to investigate the relationship between granulocyte colony-stimulating factor (G-CSF) and the condition of an ischemic stroke patient, as well as to develop the prognosis biomarker for evaluating the cure level of stroke and a method thereof.

Thus, it can be seen that the above-described conventional methods for evaluating cure level of a stroke patient have many deficiencies, are not perfect designs and need to be improved urgently.

In view of many disadvantages derived from the foregoing conventional methods for evaluating the cure level of a stroke patient, the inventor had devoted to improve and innovate, and finally, has developed successfully a prognosis biomarker for evaluating the cure level of stroke patient and a method thereof according to the invention.

### SUMMARY OF THE INVENTION

One object of the invention is to provide a method for evaluating the cure level of a stroke patient, characterized in that the method uses serum endogenous G-CSF of a stroke patient as the prognosis index.

Another object of the invention is to provide the prognosis biomarker for evaluating the cure level of a stroke patient, and a kit containing said prognosis biomarker for evaluating the cure level of a stroke patient.

The method for evaluating the cure level of a stroke patient that can achieve the above-mentioned objects according to the invention comprises following steps:
step 1: obtaining an isolated blood sample from said ischemic stroke patient;
step 2: determining the concentration of serum granulocyte colony stimulating-factor (G-CSF, SEQ ID No: 1) in said isolated blood sample;
step 3 prognosis: when said ischemic stroke patient is evaluated at initial stage as patient with serious symptom, and if lower concentration of said granulocyte colony-stimulating factor (G-CSF) in the isolated blood sample of said patient with serious symptom is determined, it can be expected that better cure level of said patient can be obtained after treatment, and this indicates better prognosis; on the contrary, when said ischemic stroke patient is evaluated at initial stage as a patient with serious symptom, and if higher concentration of said granulocyte colony-stimulating factor (G-CSF) in the isolated blood sample of said patient with serious symptom is determined, it can be expected that worse cure level of said patient may be obtained after treatment, and this indicates worse prognosis;

wherein said ischemic stroke patient is evaluated at initial stage as a patient with serious symptom in step 3, said evaluation method can evaluate the severity level of the symptom using suitable ranking method.

In a preferred embodiment of a method for evaluating the cure level of a stroke patient that can achieve the above-mentioned objects of the invention comprises following steps:
step 1: obtaining isolated sample (blood sample) from said ischemic stroke patient;
step 2: determining the concentration of serum granulocyte colony-stimulating factor (G-CSF) of said isolated blood sample;
step 3: at first, the relationship between the stroke severity of said ischemic stroke patient and the concentration of granulocyte colony-stimulating factor (G-CSF) is assessed by means of a stroke severity ranking scale, and classify said stroke severity ranking scores into low, middle, and middle-high; when said stroke severity ranking scores are middle and middle-high, it can be found that higher concentration of granulocyte colony-stimulating factor (G-CSF) in the isolated sample is obtained in step 2; on the contrary, when said stroke severity ranking score is low, lower concentration of granulocyte colony-stimulating factor (G-CSF) in the isolated sample is obtained;
step 4 prognosis: said ischemic stroke patient is treated after a suitable recovery period, and the stroke severity (cure level) of said ischemic stroke patient is assessed by means of a stroke severity ranking scale after said treatment, compared with the ranking score of said stroke patient obtained in step 3 to determine the cure level (improving level) of said patient, and then compared with the concentration of granulocyte colony-stimulating factor (G-CSF) determined in step 2; when the initial ranking score of said ischemic stroke patient is middle, and middle-high, this indicates more severe at initial stage, and if lower concentration of said granulocyte colony-stimulating factor (G-CSF) in the isolated blood sample of said middle and middle-high patient is determined, it can be expected that better cure level of said patient is obtained, and this indicates better prognosis; on the contrary, when the initial ranking score of said ischemic stroke patient is middle and middle-high, this indicate the initial stage is more severe, and if higher concentration of said granulocyte colony-stimulating factor (G-CSF) is determined in the isolated blood sample of said middle and middle-high patients, it can be expected that worse cure level of said patient can be obtained, and this indicates worse prognosis;

wherein said stroke severity ranking described in said step 3 is the United State National Institute of Health Stroke Scale (NIHSS);

wherein said stroke severity ranking score of less than or equal to 4 points (≦4) is classified as low, 5˜7 points as middle, and higher than or equal to 8 points (≧8) as middle-high.

In another preferred embodiment, the stroke severity ranking described in said step 3 is modified Ranking Scale (mRS); in said modified stroke severity ranking, classifies 0˜2 points as low, 3 points as middle, and 4˜6 points as high.

Wherein the approach for ranking stroke severity described in step 3 is only a preferred illustration, not to limit the implementation and scope of the invention, and other suitable ranking approach may be used instead. Therefore, according to the foregoing, when a ischemic stroke patient is diagnosed as middle or middle-high patient at the initial stage, as lower concentration of granulocyte colony-stimulating factor (G-CSF) is determined in the isolated blood sample, it can be expected that a better cure level of the patient can be obtained, and this indicates better prognosis; on the contrary, when a ischemic stroke patient is diagnosed as a middle or middle-high patient at initial stage, and higher concentration of granulocyte colony-stimulating factor (G-CSF) is determined in the isolated blood sample, it can be expected that a worse cure level of said patient may be obtained, and this indicates worse prognosis.

Accordingly, the prognosis biomarker and the method for evaluating cure level of a stroke patient in using the concentration of granulocyte colony-stimulating factor (G-CSF) as said biomarker in the isolated blood sample of the stroke patient, and then using said concentration of granulocyte colony-stimulating factor (G-CSF) to predict the possible cure level of the patient; wherein said concentration of granulocyte colony-stimulating factor (G-CSF) is in inverse proportion to the cure level of a stroke patient.

The invention provides further a kit containing the prognosis biomarker for evaluating the cure level of a stroke patient. Wherein the kit comprising suitable agents to practice the method according to this invention.

The invention will be illustrated by way of the following examples, but the invention is not limited by the examples described below.

These features and advantages of the present invention will be fully understood and appreciated from the following detailed description of the accompanying Drawings.

### BRIEF DESCRIPTION OF THE DRAWINGS

The following detailed description of a preferred embodiment according to the invention and the accompanying drawings thereof will be referred in order to understand further effects of its technical content and objects; wherein:

FIG. 1 shows the percentage of NIHSS improvement on patients having stroke for three months, wherein concentrations of granulocyte colony-stimulating factor (G-CSF) in each group are divided into three groups of low, middle and high concentrations; and

FIG. 2 shows the percentage of mRS on patients having stroke for three months, wherein concentrations of granulocyte colony-stimulating factor (G-CSF) in each group are divided into three groups of low, middle and high concentrations.

### DETAILED DESCRIPTION OF THE PREFERRED EMBODIMENT

### Example 1

### Material and Method

### 1. Patients Selection

Total of 75 patients were recruited from the Kuang-Tien General Hospital, Taichung, Taiwan. Patients were included if 1) the clinical diagnosis was acute ischemic stroke, 2) onset was within 24 hrs, 3) the diagnosis was confirmed on brain CT or MRI and 4) they are older than 18 years. Subjects were excluded if 1) the diagnosis was intracranial hemorrhage, or 2) there were other vascular abnormalities.

### 2. Stroke Severity Evaluation Tools

The National Institute of Health Stroke Scale (NIHSS) and modified Ranking Scale (mRS) were used as stroke severity evaluation tools.

### (1) National Institute of Health Stroke Scale, NIHSS:

The United State National Institute of Health Stroke Scale (NIHSS) is used to evaluate clinically at early stage the deficit degree of neuro function of a patient, and even assesses the prognosis, disease severity of a patient. NIHSS is consisted of 15 items, including: level of consciousness (LOC), LOC questions, LOC commands, best gaze, visual fields, facial palsy, left motor arm, right motor arm, left motor leg, right motor leg, ataxia, sensory, best language, dysarthria, extinction/inattention. Its ranking score ranges from 0 to 42, the higher the score, the more severe neurological deficit. The United State National Institute of Health Stroke Scale (NIHSS) assessment is performed by neurologist having international certification of the United State National Institute of Health Stroke Scale (NIHSS). A time period of about 5-8 minutes may be required for one complete assessment. It can assess quickly and effectively the neurological deficit degree of a stroke patient. Its ranking score ranges as followed:
less than or equal to 4 points (≦4) is low;
5˜7 points is middle;
higher than or equal to 8 points (≧8) is middle-high.

### (2) Modified Ranking Scale, mRS:

0=normal
1=no significant disability other than symptom; various daily activities and affairs can be performed
2=low disability; unable to perform all activities, but can administer affairs voluntarily without assist
3=middle disability; need some assist, but able to walk voluntarily without assist
4=middle high disability; able to walk only under assist, and unable to consider own demand without assist
5=high disability; clinically, incontinence and need consistently nursing attendance
higher than and equal to 6=death

### (3) TOTAS:

0=normal
1=small vessel
2=large atheromatous
3=cardioembolic
4=other cause
5=unspecific

Baseline evaluation was evaluated for all patients at admission, and blood samples were collected for analyzing granulocyte colony-stimulating factor (G-CSF) and other inflammatory marker, including: intercellular adhesion molecule-1 (sICAM-1), vascular cell adhesion molecule-1 (sVCAM-1), sE-selectin; other biochemical marker, fibrinogen and highly sensitive C-reactive protein (hs-CRP), blood lipids (including cholesterol, low density lipoprotein cholesterol, high density lipoprotein cholesterol, triglyceride), urea nitrogen, creatinine, total white blood cells, red blood cell, and platelet. Blood samples were collected from patients on one or two days after stroke to collect data of above-mentioned markers, and conditions of said patients were followed up continuously up to one year. In addition to assessing stroke severity of said patients by means of stroke severity ranking on Day 1-2, cure levels of 3-month recovery or 12-month recovery were evaluated on the third month and the twelfth month, respectively, thereby the relationship between granulocyte colony-stimulating factor (G-CSF) and possible cure level or prognosis could be analyzed.

3. Assays for Intercellular Adhesion Molecule-1(sICAM-1), Vascular Cell Adhesion Molecule-1(sVCAM-1) and sE-Selectin

We assayed the serum concentrations of ICAM-1, VCAM-1 and E-selectin by using commercially available enzyme-linked immunosorbent assay kits (R&D Systems, Minneapolis, Minn., USA) in accordance with the manufacturer's instructions.

4. Assays for Serum G-CSF, Fibrinogen and hs-CRP

An ELISA assay kit (R&D Systems) was used for measuring serum G-CSF levels on a 96 well microliter plate. Fibrinogen was measured by the Sysmex CA6000 coagulation analyzer with Dade Behring thrombin reagent (Dade Behring, Milton Keynes, UK). Intra-assay coefficients of variation were <4%. Highly sensitive C-reactive protein (hsCRP) was measured with BN Prospec (Dade Behring). Inter-assay and intra-assay coefficients of variation were <4% and <2% respectively, with a detection limit of 0.20 mg/L.

### 5. Statistic Assay

Data were analyzed using statistical method. A Statistical Package for the Social Sciences (SPSS; 10.5 version; Chicago, Ill., USA) was used to perform the analysis of relationship between common clinical tool markers and granulocyte colony-stimulating factor (G-CSF) and other biochemical markers.

### Example 2

### Result

Tertile function of SPSS was used to divide 75 acute stroke patients into three groups based on NIHSS/mRS scores of each patient as followed:
low NIHSS group: NIHSS≦4 points; or mRS≦2 points;
middle NIHSS group: 7 points≧NIHSS≧5 points; or mRS=3 points;
middle-high NIHSS group: NIHSS≧8 points; or 4 points≦mRS≦6 points.

Within these three groups, there was no significant variation in baseline demographic data (see Table 1) including age, sex, body mass index, smoking index, and incidence of valvular heart disease, hypertension, hyperlipidemia and diabetes mellitus. The middle-high NIHSS group had significantly higher mRS scores (p=0.031) than those in the low NIHSS groups. Accordingly, there was a positive relationship between modified Ranking Scale (mRS) score and the United State National Institute of Health Stroke Scale (NIHSS) score, and could be used as classification criteria for evaluating stroke severity.

TABLE 1
Baseline Demographics of Subjects
Grouped According to NIHSS score

| | Low | Middle | Middle-High |
|---|---|---|---|
| NIHSS score | ≦4 | 5~7 | ≧8 |
| Patient's number | n = 23 | n = 28 | n = 24 |
| Age ± SD | 69 ± 13 | 67 ± 50 | 66 ± 17 |
| Sex (Male:Female) | 12:11 | 16:12 | 10:14 |
| Body Mass Index ± SD | 24 ± 4 | 24 ± 3 | 24 ± 3 |
| Smoking Index ± SD | 17 ± 4 | 15 ± 4 | 16 ± 5 |
| Vascular Heart disease | 19% | 17% | 29% |
| Hyperlipidemia | 10% | 13% | 14% |
| Hypertension | 89% | 91% | 83% |
| Diabetes Mellitus | 18% | 30% | 29% |
| Severity of mRS score ± SD | 1.8 ± 0.5 | 2.6 ± 0.6* | 3.7 ± 1.0** |

*p < 0.05 as compared with low NIHSS group.
**p < 0.01 as compared with low NIHSS group.
mRS: modified Ranking Scale
NIHSS: National Institute of Health Stroke Scale
SD: standard deviation

Biochemical data from each NIHSS group (low, middle and middle-high groups) is summarized in table II. There is no significant difference in BUN, creatinine, total cholesterol, HDL-cholesterol, LDL-cholesterol, triglyceride, sE-selectin, sICAM-1, sVCAM-1, hs-CRP, total WBC, platelets or hemoglobin across the three groups. However, concentrations of fasting glucose, fibrinogen and G-CSF were significantly increased in the middle-high NIHSS group when compared with the other two NIHSS groups (p=0.023, p=0.016 and p=0.007, respectively). These indicated that, in severe stroke patients, concentrations of fasting glucose, fibrinogen and granulocyte colony-stimulating factor (G-CSF) were higher than those in patients in low and middle groups.

TABLE 2
Biochemical Data Grouped According to NIHSS scores

| | Low | Middle | Middle-High |
|---|---|---|---|
| NIHSS score | ≦4 | 5~7 | ≧8 |
| Patient's number | n = 23 | n = 28 | n = 24 |
| BUN (mg/dL) | 21.50 ± 12.33 | 18.74 ± 8.79 | 17.20 ± 5.69 |
| Creatinine (mg/dL) | 1.38 ± 0.83 | 1.38 ± 1.10 | 1.08 ± 0.29 |
| Total Cholesterol (mg/dL) | 164.64 ± 32.61 | 187.61 ± 42.94 | 176.81 ± 36.88 |
| Triglyceride (mg/dL) | 146.27 ± 93.00 | 163.32 ± 89.97 | 199.33 ± 92.10 |
| HDL-C (mg/dL) | 44.03 ± 0.98 | 45.47 ± 11.60 | 39.67 ± 7.88 |
| LDL-C (mg/dL) | 91.36 ± 27.86 | 108.52 ± 44.45 | 97.28 ± 41.28 |
| E-seletin (mg/dL) | 15.24 ± 13.88 | 13.61 ± 7.17 | 17.13 ± 9.52 |
| Ac sugar (mg/dL) | 132.89 ± 115.35 | 130.60 ± 69.99 | 167.00 ± 112.38* |
| ICAM-1 (mg/dL) | 344.59 ± 175.72 | 332.91 ± 172.71 | 386.27 ± 168.63 |
| VCAM-1 (mg/dL) | 548.88 ± 181.59 | 560.73 ± 278.64 | 586.18 ± 143.02 |
| Platelet (x1000/cmm) | 209.41 ± 60.32 | 215.71 ± 82.48 | 235.62 ± 94.33 |
| RBC (x10000/cmm) | 4.25 ± 0.57 | 4.30 ± 0.51 | 4.92 ± 0.61 |
| WBC (x1000/cmm) | 7.24 ± 2.58 | 7.21 ± 2.26 | 8.20 ± 2.75 |
| Fibrinogen (mg/dL) | 378.52 ± 121.37 | 353.96 ± 77.73 | 414.42 ± 99.34* |
| hs-CRP (mg/dL) | 0.53 ± 0.69 | 0.43 ± 0.46 | 0.49 ± 0.43 |
| G-CSF (μg/L) | 22.07 ± 8.32 | 22.58 ± 8.99 | 30.08 ± 13.39** |

*p < 0.05: as compared with low NIHSS group and middle NIHSS group.
**p < 0.01: as compared with low NIHSS group and middle NIHSS group.
hs-CRP: hypersensitive C-reactive protein
G-CSF: granulocyte colony stimulating-factor
sICAM-1: intercellular adhesion molecule-1
sVCAM-1: vascular cell adhesion molecule-1

Moreover, Table III shows the correlation analysis between NIHSS/mRS scores and selected biomarkers. As can be seen from Table 3, concentration of serum granulocyte colony-stimulating factor (G-CSF) has a strong correlation with modified Ranking Scale (mRS) scores and the United State National Institute of Health Stroke Scale (NIHSS) scores on the first day, as well as on third month and the twelfth month after stroke, indicating that the concentration of granulocyte colony-stimulating factor (G-CSF) on 1˜2 days after stroke could be used to evaluate the cure level on the third and twelfth months after stroke. Comparing with other biomarkers, serum G-CSF of patient is the best bio-predictor/biomarkers of stroke severity. The P value is between 1.5×10⁻⁴ to 4.4×10⁻⁵ and the Pearson correlation coefficient is between 0.350 and 0.489 (R²: 12.3˜23.9).

Further, absolute improvement values (representing stroke cure level) of the United State National Institute of Health Stroke Scale score (ΔNIHHS) and modified ranking stroke score (ΔmRS) of stroke patients that have being hospitalized about three months (as shown in Table 4); while their relative improvement values (representing stroke cure level) were calculated as shown in Table 5. Data were analyzed using multiple regression model, it could be seen that, among all biomarkers, the concentration of granulocyte colony-stimulating factor (G-CSF) was in inverse proportion to absolute improvement value (Table 4); on the other hand, the concentration of granulocyte colony-stimulating factor (G-CSF) was in inverse proportion to relative improvement value (Table 5) and had statistically significant difference (p<0.051. Accordingly, the concentration of granulocyte colony-stimulating factor (G-CSF) can be used to predict the possible cure level of a stroke patient.

TABLE 3
The power of each biomarker as a predictor of NIHSS and
mRS score status.

| | NIHSS [1-2 d] | mRS [1-2 d] | NIHSS [3 m] | mRS [3 m] | NIHSS [12 m] | mRS [12 m] |
|---|---|---|---|---|---|---|
| | PCC/P | PCC/P | PCC/P | PCC/P | PCC/P | PCC/P |
| WBC | 0.123/0.314 | 0.087/0.485 | 0.150/0.230 | 0.186/0.135 | 0.110/0.380 | 0.187/0.133 |
| Ac sugar | 0.031/0.807 | −0.068/0.601 | 0.067/0.607 | 0.066/0.609 | 0.026/0.842 | 0.101/0.435 |
| Platelet | 0.133/0.270 | 0.176/0.155 | 0.139/0.261 | 0.222/0.071 | 0.097/0.441 | 0.136/0.278 |
| Fibrinogen | 0.188/0.130 | 0.147/0.245 | 0.184/0.145 | 0.112/0.377 | 0.228/0.07 | 0.127/0.319 |
| hs-CRP | −0.133/0.289 | −0.067/0.606 | −0.098/0.450 | −0.152/0.238 | −0.044/0.737 | 0.127/0.319 |
| sICAM-1 | −0.006/0.958 | 0.001/0.998 | 0.061/0.611 | −0.029/0.807 | 0.037/0.762 | 0.020/0.871 |
| sVCAM-1 | 0.02/0.869 | 0.056/0.653 | 0.052/0.680 | 0.071/0.569 | 0.097/0.441 | 0.136/0.278 |
| sE-seletin | 0.015/0.906 | 0.047/0.710 | 0.076/0.545 | −0.008/0.948 | 0.012/0.923 | 0.037/0.771 |
| G-CSF | 0.456/4.4 × 10⁻⁵* | 0.350/2.8 × 10⁻³* | 0.489/1.5 × 10⁻⁵* | 0.421/2.5 × 10⁻⁴* | 0.434/1.5 × 10⁻⁴* | 0.395/6.4 × 10⁻⁴* |

mRS [m]: modified Ranking Scale [month]
NIHSS [m]: National Institute of Health Stroke Scale[month]
PCC: Pearson correlation coefficient
P: P value
*Pearson correlation coefficient showed significantly positive correlation and P < 0.05.
hs-CRP: hypersensitive C-reactive protein
G-CSF: granulocyte colony stimulating-factor
sICAM-1: intercellular adhesion molecule-1
sVCAM-1: vascular cell adhesion molecule-1
1-2 d: on 1 to 2 days stroke patients hospitalized; 3 m: on the third month stroke patients hospitalized;
12 m: on the twelfth month stroke patients hospitalized.

TABLE 4
The regression coefficient (β) and P value in the
multivariate linear regression analysis of absolute
improvement in NIHSS and mRS scores (ΔNIHSS/ΔmRS)

| | |
|---|---|
| ΔNIHSS as the | ΔmRS as the |
| dependent | dependent |
| variable | variable |

| | β | P | β | P |
|---|---|---|---|---|
| G-CSF | −0.453 | 0.008 | −0.291 | 0.032 |
| fibrinogen | 0.0113 | 0.440 | 0.0160 | 0.220 |
| Ac sugar | −0.135 | 0.318 | −0.170 | 0.157 |
| age | −0.165 | 0.254 | −0.151 | 0.243 |
| sex | −0.042 | 0.742 | −0.010 | 0.933 |
| TOAST | −0.188 | 0.180 | −0.190 | 0.131 |
| NIHSS (1^{st} or 2^{nd} day | 0.115 | 0.492 | −0.302 | 0.031 |
| after stroke) | | | | |

ΔNIHSS: NIHSS scores of the first day − NIHSS scores of the third month
ΔmRS: mRS scores of the first day − mRS scores of the third month
P: P value
G-CSF: granulocyte colony stimulating-factor
TOAST: Trial of ORG 10172 in Acute Stroke Treatment (TOAST) classification of stroke subtype

TABLE 5
The regression coefficient (β) and P value in the
multivariate linear regression analysis of relative improvement
in NIHSS and mRS scores (Δ^{R}NIHSS/Δ^{R}mRS)

| | |
|---|---|
| Δ^{R}NIHSS as the | Δ^{R}mRS as the |
| dependent | dependent |
| variable | variable |

| | β | P | β | P |
|---|---|---|---|---|
| G-CSF | −0.453 | 0.008 | −0.335 | 0.032 |
| fibrinogen | −0.135 | 0.318 | −0.185 | 0.146 |
| Ac sugar | 0.113 | 0.440 | 0.157 | 0.251 |
| age | −0.165 | 0.254 | −0.184 | 0.173 |
| sex | −0.042 | 0.742 | 0.018 | 0.883 |
| TOAST | −0.188 | 0.180 | −0.260 | 0.049 |
| NIHSS (1^{st} or 2^{nd} day | 0.115 | 0.492 | −0.105 | 0.499 |
| after stroke) | | | | |

Δ^{R}NIHSS: [(NIHSS scores of the first day − NIHSS scores of the third month)/NIHSS scores of the first day] × 100%
Δ^{R}mRS: [(mRS scores of the first day − mRS scores of the third month)/mRS scores of the first day] × 100%
NIHSS: National Institute of Health Stroke Scale
P: P value
β: the regression coefficient
G-CSF: granulocyte colony stimulating-factor
TOAST: Trial of ORG 10172 in Acute Stroke Treatment (TOAST) classification of stroke subtype

Stroke patients (at the stroke day 1˜2) in low, middle, and middle-high groups of the United State National Institute of Health Stroke Scale (NIHSS) were divided into three subgroups based on the concentrations of serum granulocyte colony-stimulating factor (G-CSF) of their isolated blood sample:
lower concentration subgroup: G-CSF<18.4 μg/L;
middle concentration subgroup: 18.4 μg/L≦G-CSF≦27.0 μg/L;
middle-high concentration subgroup: G-CSF>27.0 μg/L.

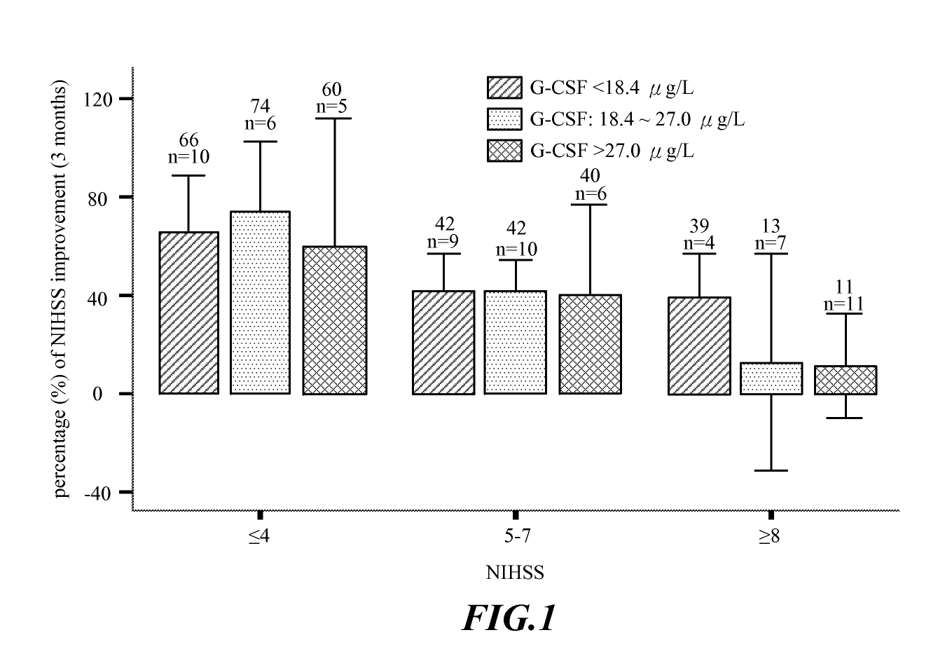

As showed in FIG. 1, three months after the stroke, the percentage improvement in NIHSS score was good in all three G-CSF subgroups of the low NIHSS score group, with scores of 66%, 74% and 60% respectively but no significant difference between them (p>0.05). In the middle NIHSS score group, there was also no significant difference in the percentage change between the G-CSF subgroups (42%, 42% and 40%, respectively; p>0.05). In the middle-high NIHSS score group however, the lower G-CSF subgroup had greater improvements in neurologic deficits compared with the middle and higher G-CSF subgroups (39% versus 13% and 11%, respectively; p<0.05). Obviously, in the middle-high group, compared with those two subgroups of middle, and high concentrations, the subgroup of lower granulocyte colony-stimulating factor (G-CSF) concentration demonstrated more improvement of stroke cure level with respect of neurological deficit. These indicates that, when lower concentration of granulocyte colony-stimulating factor (G-CSF) is detected in the middle-high stroke patient, better cure level and hence better prognosis can be expected.

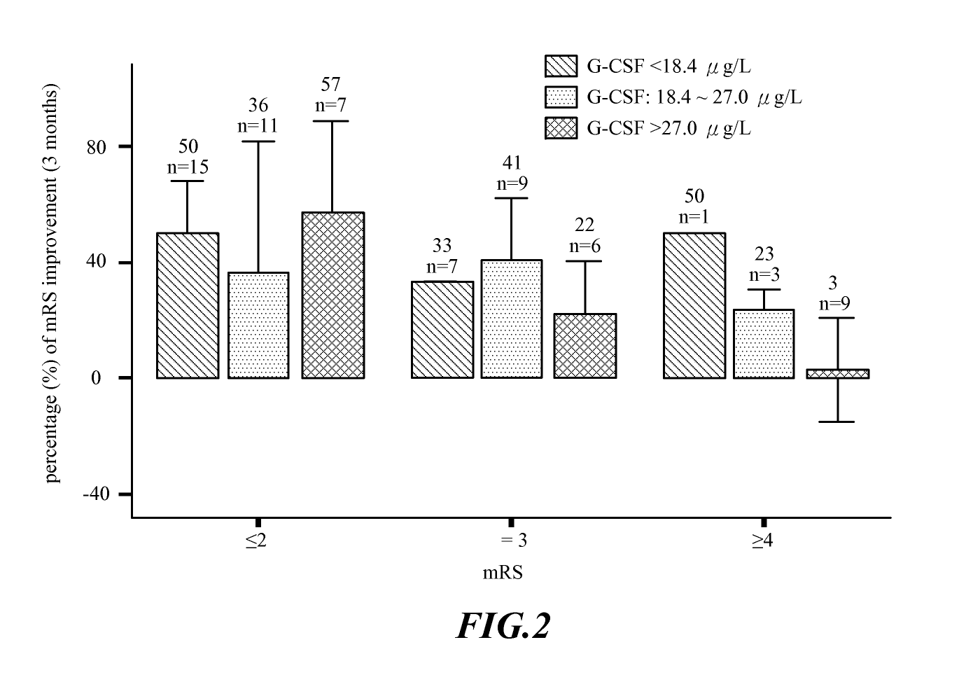

When using the mRS score instead, a similar result was seen. FIG. 2 shows that the percentage improvement at 3 months after the stroke was no different across the three G-CSF subgroups in the lower and middle NIHSS groups (lower NIHSS group: 50%, 36% and 57%, p>0.05; middle NIHSS group: 33% 41% and 22%, p>0.05 respectively). Similarly, in the middle-high mRS group, the lower concentration G-CSF subgroups showed greater improvement in neurologic deficit and mRS scores (50% versus 23% and 3% respectively, p<0.05). This indicates that, when lower concentration of granulocyte colony-stimulating factor (G-CSF) is detected in a middle-high stroke patient, better cure level and hence better prognosis can be expected.

These data shows that, according to the NIHSS and mRS scoring systems, the concentration of endogenous G-CSF can be used to predict the possible cure level of a patient 3 months after the stroke. In middle and middle-high groups according to the NIHSS and mRS scoring systems, high concentration of serum granulocyte colony-stimulating factor (G-CSF) subgroup correlates apparently with the lower percentage improvement of the NIHSS and mRS, that is, in the middle-high stroke patient, if a lower concentration of granulocyte colony-stimulating factor (G-CSF) is detected, it can be expected that said middle-high stroke patient may have better cure level (improvement level) thereafter, and hence better prognosis; on the contrary, in the middle and middle-high groups of the NIHSS and mRS scores, if a higher concentration of granulocyte colony-stimulating factor (G-CSF) is detected in the middle-high stroke patient, a worse cure level (improvement level) and hence worse prognosis may be expected in the middle-high stroke patient.

Besides, the present invention also measured various inflammatory markers to assess for a relationship with the severity of acute ischemic stroke. Although no significant relationship was seen, an increasing trend in sICAM-1 and sVCAM-1 was noted in the middle-high NIHSS score groups. However, increases in fasting blood sugar, serum fibrinogen and G-CSF correlated significantly with the severity of acute ischemic stroke. Therefore, present invention provides that serum levels of endogenous G-CSF are a better and more sensitive predictive biomarker of severity in acute ischemic stroke than hs-CRP, sE-selection, sICAM-1 and sVCAM-1. Analyzing the serum G-CSF concentration of patient, it can be used to validate the prognosis of acute ischemic stroke.

The prognosis biomarker and the method for evaluating the cure level of a stroke patient provided according to the invention have following advantage over other conventional techniques:

By way of statistical analysis of data, the invention can provide reference markers more rapidly and more accurately for a physician to evaluate the recovery status after stroke treatment, thereby not only can assist the evaluation of neurological regulation condition after the stroke, but also can predict the recovery condition of the patient one year after the stroke.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

What is claimed is:

1. A method for evaluating the cure level of a stroke patient, comprising following steps:
step 1: obtaining isolated blood sample from said ischemic stroke patient;
step 2: determining the concentration of serum granulocyte colony stimulating-factor (G-CSF) of said isolated blood sample;
step 3 prognosis: when said ischemic stroke patient is diagnosed at initial stage as a patient with serious symptom, and if lower concentration of granulocyte colony-stimulating factor (G-CSF) is determined in the isolated blood sample of said patient with serious symptom, better cure level can be expected in said patient after treatment, and this indicates better prognosis; on the contrary, when said ischemic stroke patient is diagnosed at initial stage as a patient with serious symptom, and if higher concentration of granulocyte colony-stimulating factor (G-CSF) is determined in the isolated blood sample, worse cure level can be expected in said patient after treatment, this indicates worse prognosis.

2. A method for evaluating the cure level of a stroke patient as recited in claim 1, wherein said granulocyte colony-stimulating factor (G-CSF) has a sequence as depicted in SEQ ID No: 1.

3. A method for evaluating the cure level of a stroke patient, comprising following steps:
step 1: obtaining isolated blood sample from said ischemic stroke patient;
step 2: determining the concentration of serum granulocyte colony-stimulating factor (G-CSF) of said isolated blood sample;
step 3: at first, the relationship between the stroke severity of said ischemic stroke patient and the concentration of granulocyte colony-stimulating factor (G-CSF) is assessed by means of a stroke severity ranking scale, and classify said stroke severity ranking scores into low, middle, and middle-high; when said stroke severity ranking scores are middle and middle-high, it can be found that higher concentration of granulocyte colony-stimulating factor (G-CSF) in the isolated sample is obtained in step 2; on the contrary, when said stroke severity ranking score is low, lower concentration of granulocyte colony-stimulating factor (G-CSF) in the isolated sample is obtained;
step 4 prognosis: said ischemic stroke patient is treated after a suitable recovery period, and the stroke severity of said ischemic stroke patient is assessed by means of a stroke severity ranking scale after said treatment, compared with the ranking score of said stroke patient obtained in step 3 to determine the cure level of said patient, and then compared with the concentration of granulocyte colony-stimulating factor (G-CSF) determined in step 2;
when the initial ranking score of said ischemic stroke patient is middle, and middle-high, this indicates more severe at initial stage, and if lower concentration of said granulocyte colony-stimulating factor (G-CSF) in the isolated blood sample of said middle and middle-high patient is determined, it can be expected that better cure level of said patient is obtained, and this indicates better prognosis; on the contrary, when the initial ranking score of said ischemic stroke patient is middle and middle-high, this indicate the initial stage is more severe, and if higher concentration of said granulocyte colony-stimulating factor (G-CSF) is determined in the isolated blood sample of said middle and middle-high patients, it can be expected that worse cure level of said patient can be obtained, and this indicates worse prognosis.

4. A method for evaluating the cure level of a stroke patient as recited in claim 3, where said stroke severity ranking in said step 3 and step 4 is the United State National Institute of Health Stroke Scale (NIHSS).

5. A method for evaluating the cure level of a stroke patient as recited in claim 4, wherein said stroke severity ranking score classifies less than or equal to 4 points as low, 5˜7 points as middle, and higher than or equal to 8 points as middle-high.

6. A method for evaluating the cure level of a stroke patient as recited in claim 3, wherein said stroke severity ranking in said step 3 and step 4 is a modified Ranking Scale (mRS).

7. A method for evaluating the cure level of a stroke patient as recited in claim 6, wherein said stroke severity ranking score classifies 0˜2 points as low, 3 points as middle, and 4˜6 points as middle-high.

8. A method for evaluating the cure level of a stroke patient as recited in claim 3, wherein said granulocyte colony-stimulating factor (G-CSF) has a sequence as depicted in SEQ ID No:1.

9. A prognosis biomarker for evaluating the cure level of a stroke patient, characterized in that the concentration of serum granulocyte colony-stimulating factor (G-CSF) of isolated blood sample of an ischemic stroke patient is used as said prognosis biomarker.

10. A prognosis biomarker for evaluating the cure level of a stroke patient as recited in claim 9, wherein said granulocyte colony-stimulating factor (G-CSF) has a sequence as depicted in SEQ ID No: 1.

11. A prognosis biomarker for evaluating the cure level of a stroke patient as recited in claim 9, wherein said concentration of granulocyte colony-stimulating factor (G-CSF) is in inverse proportion to the possible cure level/prognosis of said ischemic stroke patient.

12. A prognosis biomarker for evaluating the cure level of a stroke patient as recited in claim 9, wherein said prognosis biomarker can further be prepared as a kit for evaluating the cure level of a stroke patient.


SEQUENCE LISTING (1 sequence)

SEQ ID NO: 1 - DNA, 2379 bp, Homo sapiens
  gene (1)...(2379)<s300> Genbank/ NC_0000172008-03-03
aaaacagcccggagcctgcagcccagccccacccagacccatggctggacctgccacccagagccccatgaagctgatgggtgagtgtcttggcccaggatgggagagccgcctgccctggcatgggagggaggctggtgtgacagaggggctggggatccccgttctgggaatggggattaaaggcacccagtgtccccgagagggcctcaggtggtagggaacagcatgtctcctgagcccgctctgtccccagccctgcagctgctgctgtggcacagtgcactctggacagtgcaggaagccacccccctgggccctgccagctccctgccccagagcttcctgctcaagtgcttagagcaagtgaggaagatccagggcgatggcgcagcgctccaggagaagctggtgagtgaggtgggtgagagggctgtggagggaagcccggtggggagagctaagggggatggaactgcagggccaacatcctctggaagggacgtgggagaatattaggagcagtggagctggggaaggctgggaagggacttggggaggaggaccttggtggggacagtgctcgggagggctggctgggatgggagtggaggcattacattcaggagaaagggcaagggcccctgtgagatcagagagtgggggtgcagggcagagaggaactgaacagcctggcaggacatggagggaggggaaagaccagagagtcggggaggacccgggaaggagcggcgacccggccatggcgagtctcactcagcatccttccatccccagtgtgccacctacaagctgtgccaccccgaggagctggtgctgctcggacactctctgggcatcccctgggctcccctgagcagctgccccagccaggccctgcagctggtgagtgtcaggaaaggataaggctaatgaggagggggaaggagaggaggaacacccatgggctcccccatgtctccaggttccaagctgggggcctgacgtatctcaggcagcaccccctaactcttccgctctgtctcacaggcaggctgcttgagccaactccatagcggccttttcctctaccaggggctcctgcaggccctggaagggatctcccccgagttgggtcccaccttggacacactgcagctggacgtcgccgactttgccaccaccatctggcagcaggtgagccttgttgggcagggtggccaaggtcgtgctggcattctgggcaccacagccaggcctgtgtatgggccctgtccatgctgtcacccccagcatttcctcatttgtaataacgcccactcagaagggcccaaccactgatcacagctttcccccacagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagccctgagccaagccctccccatcccatgtatttatctctatttaatatttatgtctatttaagcctcatatttaaagacagggaagagcagaacggagccccaggcctctgtgtccttccctgcatttctgagtttcattctcctgcctgtagcagtgagaaaaagctcctgtcctcccatcccctggactgggaggtagataggtaaataccaagtatttattactatgactgctccccagccctggctctgcaatgggcactgggatgagccgctgtgagcccctggtcctgagggtccccacctgggacccttgagagtatcaggtctcccacgtgggagacaagaaatccctgtttaatatttaaacagcagtgttccccatctgggtccttgcacccctcactctggcctcagccgactgcacagcggcccctgcatccccttggctgtgaggcccctggacaagcagaggtggccagagctgggaggcatggccctggggtcccacgaatttgctggggaatctcgtttttcttcttaagacttttgggacatggtttgactcccgaacatcaccgacgcgtctcctgtttttctgggtggcctcgggacacctgccctgcccccacgagggtcaggactgtgactctttttagggccaggcaggtgcctggacatttgccttgctggacggggactggggatgtgggagggagcagacaggaggaatcatgtcaggcctgtgtgtgaaaggaagctccactgtcaccctccacctcttcaccccccactcaccagtgtcccctccactgtcacattgtaactgaacttcaggataataaagtgtttgcctcca